(19) United States
(12) Patent Application Publication
(10) Pub. No.: US 2012/0322996 A1
(43) Pub. Date: Dec. 20, 2012

(54) Novel Method for the Preparation of Stavudine Polymorphic Form I and Form II

(76) Inventors: Gianluca Di Lernia, Paderno D'Adda (LC) (IT); Alfredo Mancini, Spinadesco (CR) (IT); Franco Mancini, Cremona (IT); Maurizio Serra, Arzago D'Adda (BG) (IT); Domenico Vergani, Biassono (MI) (IT)

(21) Appl. No.: 12/160,629

(22) PCT Filed: Feb. 26, 2007
(86) PCT No.: PCT/EP07/51812

§ 371 Date: Jul. 11, 2008

(30) Foreign Application Priority Data
Feb. 28, 2006 (IT) MI2006A000360

(51) Int. Cl. C07H 1/00

(52) U.S. Cl. 536/28.54

(57) ABSTRACT

A novel method for the preparation of stavudine polymorphic form I and form II is described. 5′-acetate-2′,3′-diacetyl-5-methyluridine is reacted with catalytic amounts of sodium methoxide in a C₁-C₄ alcoholic solvent, resulting in crude stavudine form II. Crude stavudine form II can be converted into polymorphic stavudine form I by slurry at reflux in isopropanol, without isolating or purifying the crude stavudine form II.

A novel method for the preparation of stavudine polymorphic form I and form II is described; it's prepared starting from 5′-acetate-2′,3′-diacetyl-5-methyluridine, whose chemical formula is reported here-below:

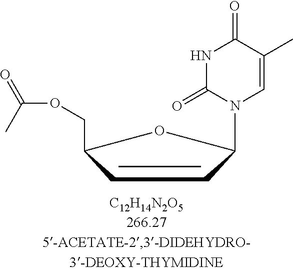

The 5′-acetate-2′,3′-diacetyl-5-methyluridine, by reaction with sodium methoxide in catalytic amounts, gives crude stavudine form II. Crude stavudine form II (which does not need to be isolated or purified), may be converted into stavudine polymorphic form I by slurry at reflux in isopropanol.

### BACKGROUND OF THE INVENTION

The here-below reported compound,

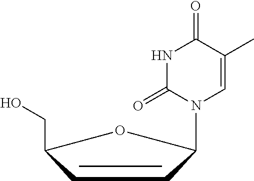

whose International Denomination is “stavudine” (D4T), is a well know active principle with antiviral action.

Several processes for preparing stavudine are described in literature, for example in: EP-A-0340778, EP-A-0493602, EP-A-0501511, US20010039342 and in Mansuri et al., J. Org. Chem. 1989, 54, 4780-4785 and in Classon et al., Acta Chem. Scand., B36, 1982, 251.

Methods of preparation of stavudine are for instance disclosed in EP0749969, Harte, W. E., et. al., Biochemical and Biophysical Research Comm., 175(1), pp 298-304 (1991) and Ghandi, R. B., Bogardus, J. B., et. al., International Journal of Pharmaceutics 201 (2000) 221-237. Three solid state forms of stavudine are reported in literature, namely form I, form II and form III. In particular, in the European patent application EP0749969, the conversion method of stavudine from one polymorphic form to another one is described.

### DESCRIPTION OF THE INVENTION

The present invention relates to the preparation of stavudine polymorphic form I and form II from 5′-acetate-2′,3′-diacetyl-5-methyluridine by the following route:

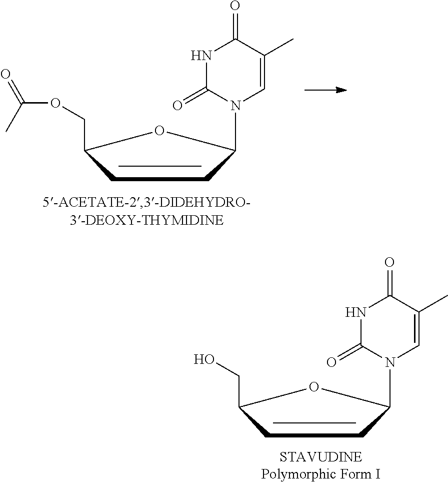

This process is very easy and fast, doesn't involve any isolation or purification of the intermediates, and allows to recover stavudine in very pure form.

More in details, the present invention relates to a novel method for the preparation of stavudine wherein:

a) the reaction of 5′-acetate-2′,3′-diacetyl-5-methyluridine with catalytic amounts of sodium methoxide;
b) the resulting crude stavudine is identified as form II;
c) and, optionally, the resulting stavudine form II is suspended and stirred at reflux in isopropanol to give pure stavudine polymorphic form I.

According to a first preferred embodiment, the method is performed without any intermediate isolation or purification before the filtration of stavudine polymorphic form I or form II.

The 5′-acetate-2′,3′-diacetyl-5-methyluridine is suspended in a C₁-C₄ alcohol at a preferred temperature of 0+30° C., especially at 15+25° C. The alcohol is preferably methanol.

Sodium methoxide is added at 0+30° C., especially at 15+25° C. In a preferred embodiment of the reaction, for each mole of 5′-acetate-2′,3′-diacetyl-5-methyluridine, 0.2+0.01 moles, especially 0.05+0.01, more preferably about 0.02 moles of sodium methoxide are used.

The suspension is heated to the reflux temperature of the alcohol; that is 40+65° C., especially to 50+65° C., more especially to 50° C., in the case of methanol.

After 3+10 hours, especially 5+8 hours, the solution obtained is cooled then the solvent is distilled off; at this point, a preliminary analysis identified on the stavudine in suspension as polymorphic form II pure.

Isopropanol is added and the suspension is distilled again under vacuum.

Isopropanol is added to give a suspension:
in order to obtain the form I, the suspension is heated at reflux for 2+15 hours, especially for 2+4 hours then is cooled to 0+30° C., especially at 15+25° C. and isopropanol is added; the suspension is stirred and filtered at 0+30° C., especially at 15+25° C.; the cake is washed by isopropanol and pure stavudine polymorphic form I obtained is dried under vacuum;
in order to isolate the form II, the suspension is stirred at 0+30° C., especially at 15+25° C. and isopropanol is added; the suspension is stirred and filtered at 0+30° C., especially at 15+25° C.; the cake is washed by isopropanol and pure stavudine polymorphic form II obtained is dried under vacuum.

Otherwise pure stavudine form I from pure stavudine form II is obtained by the suspension of pure stavudine form II (before isolated) in isopropanol is stirred at 0+30° C., especially at 15+25° C. and other isopropanol is added. The suspension is stirred and filtered at 0+30° C., especially at 15+25° C. The cake is washed by isopropanol and pure stavudine polymorphic form I obtained is dried under vacuum.

The object of the present invention is therefore represented by a process for the preparation of stavudine form I or II which comprises reacting 5′-acetate-2′,3′-diacetyl-5-methyluridine with catalytic amounts of sodium methoxide, thereby obtaining stavudine form II.

The reaction of 5′-acetate-2′,3′-diacetyl-5-methyluridine with sodium methoxide is performed in a C₁-C₄ alcohol, preferably in methanol, at the reflux temperature of the alcohol; that is, normally at 40+65° C., preferably at 50+65° C., more preferably at 50° C.

According to an embodiment of the invention, 0.2+0.01 moles of sodium methoxide, preferably 0.05+0.01 moles, even more preferably about 0.02 moles, are used per mole of 5′-acetate-2′,3′-diacetyl-5-methyluridine.

According to another embodiment, the reaction mixture is heated for 3+10 hours, preferably 5+8 hours.

The thus obtained stavudine form II can be isolated upon suspension in isopropanol under stirring, and subsequent filtration.

Otherwise, it can be converted into form I either after isolation or without the necessity of being isolated or purified. This can be accomplished by suspending the same in isopropanol and then heating the suspension to reflux; according to one embodiment of the invention, the suspension is heated to reflux for at least 2 hours, preferably for 2+15 hours, more preferably for 2+4 hours; the accordingly heated suspension is then cooled to 0+30° C., preferably to 15+25° C.; isopropanol is then added to the cooled suspension, which is then stirred and subsequently filtered at 0+30° C., preferably at 15-25° C.

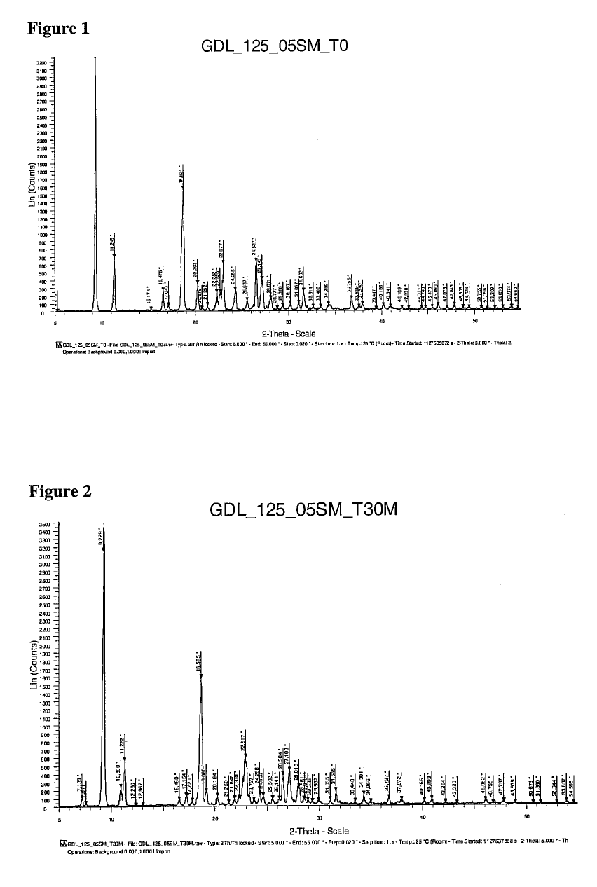

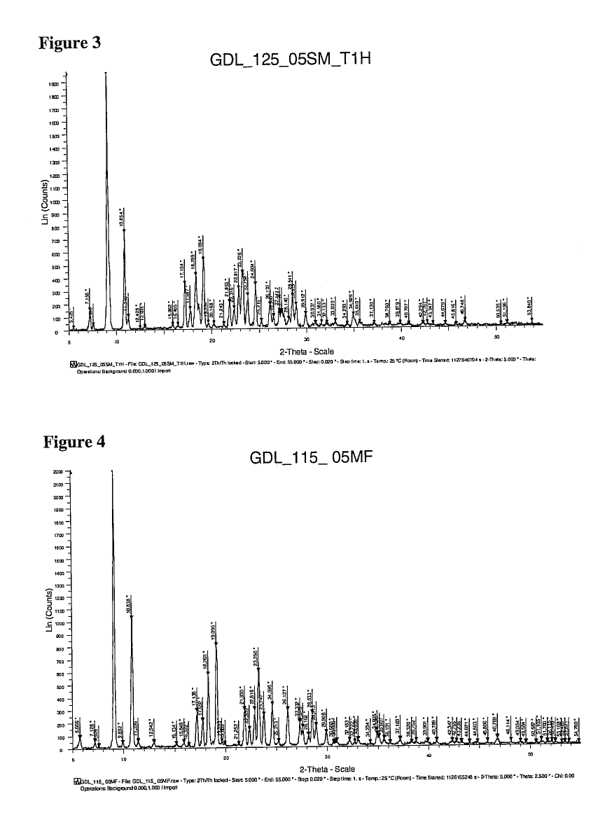

A polymorphic study was performed in order to quantitatively evaluate the kinetic conversion of form II into form I; the data was obtained by packing a sampler specimen onto a glass slide with a 0.2 mm sample well, which was analyzed using a Bruker AXS D8 Avance diffractometer. The sample was scanned from 5° to 55° 2θ, step scan Δ2θ=0.02°, t=1 s. The crude stavudine was suspended in isopropanol; at this point the powder was characterized by IR spectroscopy and by X-ray diffraction pattern, which has been repeated at 30, 60 and 120 minutes, with the following results:
at zero minutes at reflux, stavudine is identified as 100% form II (FIG. 1);
after 30 minutes at reflux in isopropanol, the conversion is monitored and characterized by X-ray diffraction patterns and IR: stavudine is identified as about 95% form II and 5% form I (FIG. 2);
after one hour at reflux in isopropanol, the conversion is monitored and characterized by X-ray diffraction patterns and IR: stavudine is identified as about 10% form II and 90% form I (FIG. 3);
after two hours at reflux in isopropanol, the conversion is completed: it is identified as 100% form I (FIG. 4).

The kinetic conversion is summarized in the table below:

| Figure | Slurry time | Form I | Form II |
|---|---|---|---|
| 1 | 0 seconds | 0% | 100% |
| 2 | 30 minutes | 5% | 95% |
| 3 | 60 minutes | 90% | 10% |
| 4 | 120 minutes | 100% | 0% |

Within the terms of the present invention the expression “the product obtained by reacting 5′-acetate-2′,3′-diacetyl-5-methyluridine with sodium methoxide” is preferably intended to indicate stavudine form II but may also have other meanings as for instance a mixture of stavudine forms I and II.

As it can be appreciated by the following examples, which are just illustrative and not limitative of the invention, the present process allows to reduce considerably the reaction salts, the work-up times, and does not involve the isolation and crystallization of pure polymorphic form II which is then converted into form I.

### EXAMPLES

### 1) Stavudine Polymorphic Form I

150 g of 5′-acetate-2′,3′-diacetyl-5-methyluridine (0.5622 mol) are suspended at 25° C. in 150 ml of methanol (10 volumes on 5′-acetate-2′,3′-diacetyl-5-methyluridine). At 25° C. 22.2 ml of sodium methoxide solution 0.5M in methanol (0.0111 mol) are added. The suspension is heated to reflux for 7 hours. The solution obtained is cooled to room temperature and 7.5 g of charcoal are added. The suspension is stirred for 30 minutes at room temperature and is filtered at room temperature. The solution obtained is distilled at 60° C. under vacuum to oil. To the oil obtained 100 ml of isopropanol (0.67 volumes on 5′-acetate-2′,3′-diacetyl-5-methyluridine) are added and the solution is distilled again at 60° C. under vacuum. To the suspension obtained 450 ml of isopropanol (3 volumes on 5′-acetate-2′,3′-diacetyl-5-methyluridine) are added and the suspension is heated to reflux for 14-16 hours. The suspension is cooled to 25° C. and 600 ml of isopropanol (3 volumes on 5′-acetate-2′,3′-diacetyl-5-methyluridine) are added and the suspension is stirred at 25° C. for 30 minutes. The suspension is filtered and the cake is washed three times with isopropanol (200 ml×3). The wet stavudine is dried under vacuum at 50° C. for 10-14 hours. Pure stavudine polymorphic form I: 103 g.

### 2) Stavudine Polymorphic Form I

150 g of 5′-acetate-2′,3′-diacetyl-5-methyluridine (0.5622 mol) are suspended at 25° C. in 150 ml of methanol (10 volumes on 5′-acetate-2′,3′-diacetyl-5-methyluridine). At 25° C. 55.5 ml of sodium methoxide solution 0.5M in methanol (0.0277 mol) (0.05 eq on 5′-acetate-2′,3′-diacetyl-5-methyluridine) are added. The suspension is heated to 50° C. for 7 hours. The solution obtained is cooled to room temperature and 7.5 g of charcoal are added. The suspension is stirred for 30 minutes at room temperature and is filtered at room temperature. The solution obtained is distilled at 60° C. under vacuum to oil. To the oil obtained 100 ml of isopropanol (0.67 volumes on 5′-acetate-2′,3′-diacetyl-5-methyluridine) are added and the solution is distilled again at 60° C. under vacuum. To the suspension obtained 450 ml of isopropanol (3 volumes on 5′-acetate-2′,3′-diacetyl-5-methyluridine) are added and the suspension is heated to reflux for 2-3 hours. The suspension is cooled to 25° C. and 600 ml of isopropanol (3 volumes on 5′-acetate-2′,3′-diacetyl-5-methyluridine) are added and the suspension is stirred at 25° C. for 30 minutes. The suspension is filtered and the cake is washed three times with isopropanol (200 ml×3). The wet stavudine is dried under vacuum at 50° C. for 10-14 hours. Pure stavudine polymorphic form I: 99 g

### 3) Stavudine Polymorphic Form II

150 g of 5′-acetate-2′,3′-diacetyl-5-methyluridine (0.5622 mol) are suspended at 25° C. in 150 ml of methanol (10 volumes on 5′-acetate-2′,3′-diacetyl-5-methyluridine). At 25° C. 55.5 ml of sodium methoxide solution 0.5M in methanol (0.0277 mol) (0.05 eq on 5′-acetate-2′,3′-diacetyl-5-methyluridine) are added. The suspension is heated to 50° C. for 5 hours. The solution obtained is cooled to room temperature and 7.5 g of charcoal are added. The suspension is stirred for 30 minutes at room temperature and is filtered at room temperature. The solution obtained is distilled at 60° C. under vacuum to oil.

To the oil obtained 100 ml of isopropanol (0.67 volumes on 5′-acetate-2′,3′-diacetyl-5-methyluridine) are added and the solution is distilled again at 60° C. under vacuum. To the suspension obtained 450 ml of isopropanol (3 volumes on 5′-acetate-2′,3′-diacetyl-5-methyluridine) are added and the suspension; the suspension is stirred for 30 minutes at room temperature. 600 ml of isopropanol (3 volumes on 5′-acetate-2′,3′-diacetyl-5-methyluridine) are added and the suspension is stirred at room temperature for other 30 minutes. The suspension is filtered and the cake is washed three times with isopropanol (200 ml×3). The wet stavudine is dried under vacuum at 50° C. for 10-14 hours. Pure stavudine polymorphic form II: 98 g

### 4) Stavudine Polymorphic Form 1 from Stavudine Polymorphic Form II

To 100 g of stavudine polymorphic form II isolated 300 ml of isopropanol (3 volumes) are added and the suspension is heated to reflux for 2-4 hours. The suspension is cooled to 25° C. and 300 ml of isopropanol (3 volumes on 5′-acetate-2′,3′-diacetyl-5-methyluridine) are added and the suspension is stirred at 25° C. for 30 minutes. The suspension is filtered and the cake is washed three times with isopropanol (200 ml×3). The wet stavudine is dried under vacuum at 50° C. for 10-14 hours. Pure Stavudine polymorphic form I: 95 g

1. A process for the preparation of stavudine form I or II comprising reacting 5′-acetate-2′,3′-diacetyl-5-methyluridine with catalytic amounts of sodium methoxide.

2. A process according to claim 1, said process comprising introducing 0.2 to 0.01 moles of sodium methoxide/mole of 5′-acetate-2′,3′-diacetyl-5-methyluridine.

3. A process according to claim 2, said process comprising introducing about 0.02 moles of sodium methoxide/mole of 5′-acetate-2′,3′-diacetyl-5-methyluridine.

4. A process according to claim 1, wherein the reaction of 5′-acetate-2′,3′-diacetyl-5-methyluridine with sodium methoxide is performed in a C₁-C₄ alcohol.

5. A process according to claim 4, wherein said alcohol is methanol.

6. A process according to claim 4, wherein said reaction is performed at the reflux temperature of the alcohol.

7. A process according to claim 6, wherein said reaction is performed at 40 to 65° C.

8. A process according to claim 1, wherein the 5′-acetate-2′,3′-diacetyl-5-methyluridine and sodium methoxide form a reaction mixture and the reaction mixture is heated for 3 to 10 hours.

9. A process for the preparation of stavudine form I according to claim 1, wherein product obtained by reacting 5′-acetate-2′,3′-diacetyl-5-methyluridine with sodium methoxide is suspended in isopropanol and the resulting suspension is heated to reflux.

10. A process according to claim 9, wherein the suspension is heated to reflux for at least 2 hours.

11. A process according to claim 9, wherein the heated suspension is cooled to 0 to 30° C.

12. A process according to claim 11, wherein isopropanol is added to the cooled suspension.

13. A process according to claim 12, wherein the cooled suspension is stirred and subsequently filtered at 0 to 30° C.

14. A process according to claim 9, wherein the product obtained by reacting 5′-acetate-2′,3′-diacetyl-5-methyluridine with sodium methoxide is stavudine form II or a mixture of stavudine forms I and II.

15. A process according to claim 9, wherein the product obtained by reacting 5′-acetate-2′,3′-diacetyl-5-methyluridine with sodium methoxide is neither isolated nor purified before being suspended in isopropanol.

16. A process for the isolation of stavudine form I according to claim 1, wherein the product obtained by reacting 5′-acetate-2′,3′-diacetyl-5-methyluridine with sodium methoxide is suspended in isopropanol under stirring.

17. A process according to claim 2, wherein said process comprises introducing from 0.05 to 0.01 moles of sodium methoxide/mole of 5′-acetate-2′,3′-diacetyl-5-methyluridine.

18. A process according to claim 7, wherein said reaction is performed at 50 to 65° C.

19. A process according to claim 7, wherein said reaction is performed at 50° C.

20. A process according to claim 8, wherein the reaction mixture is heated for 5 to 8 hours.

21. A process according to claim 10, wherein the suspension is heated to reflux for 2 to 15 hours.

22. A process according to claim 10, wherein the suspension is heated to reflux for 2 to 4 hours.

23. A process according to claim 1 wherein the heated suspension is cooled to 15 to 25° C.

24. A process according to claim 13, wherein the cooled suspension is stirred and subsequently filtered at 15 to 25° C.
